(19) United States
(12) Patent Application Publication
(10) Pub. No.: US 2015/0272699 A1
(43) Pub. Date: Oct. 1, 2015

(54) TOOL FOR INSERTION AND EXTRACTION OF IMPLANTABLE MARKERS

(71) Applicant: BLUE BELT TECHNOLOGIES, INC., PLYMOUTH, MN (US)

(72) Inventors: James Moody, Pittsburgh, PA (US); Benjamin Oliver McCandless, Pittsburgh, PA (US)

(21) Appl. No.: 14/669,600

(22) Filed: Mar. 26, 2015

Related U.S. Application Data
(60) Provisional application No. 61/970,713, filed on Mar. 26, 2014.

(51) Int. Cl. A61B 19/00

(52) U.S. Cl. CPC A61B 19/5244; A61B 19/54; A61B 2019/5491; A61B 2019/5487; A61B 2019/5261

(57) ABSTRACT

Embodiments of a system and method for insertion and extraction of a sensor assembly are generally described herein. A system can include an insertion tool comprising: a grip portion including a locking slot, and an extension portion extending distally from the grip portion to a distal end of the insertion tool. The system can include a sensor assembly detachably coupled to the distal end of the extension portion, the sensor assembly including a head portion and an anchoring portion, the anchoring portion configured to attach to a bone and a cable running through the extension portion and attached to the sensor assembly, wherein the cable is secured to the grip portion of the insertion tool at the locking slot, wherein the grip portion and the extension portion are configured to be detached from the sensor assembly and the cable after the sensor assembly is attached to the bone.

### CLAIM OF PRIORITY

This patent application claims the benefit of priority of U.S. Provisional Patent Application Ser. No. 61/970,713, titled “Tool for Insertion and Extraction of Implantable Markers,” filed on Mar. 26, 2014, which is hereby incorporated by reference herein in its entirety.

### TECHNICAL FIELD

This document relates generally, but not by way of limitation, to a tool for inserting and extracting implantable sensors, such as the ones that are used as reference points in the field of computer-aided and navigated surgery.

### BACKGROUND

The use of implantable markers and tracked sensors, which enable the tracking of internal structures in relation to surgical tools used during a procedure, has developed in recent years and now allows members of a surgical team to plan and execute procedures with the aid of computerized displays of any tools or target patient anatomy involved. Implantable markers are especially important in minimally invasive orthopedic surgical applications because the surgical team is often unable to directly see the patient's anatomy due to the small size of the incision.

Typical implantable markers comprise a sensor assembly including some structure, such as a screw or press-fit configuration, for anchoring the sensor assembly to the target area of a patient. A sensor located within the anchor tracks the location of the marker in the surgical field, and a cable, for communicating information from the sensor, runs from the marker to a computer processor where the positional information relayed is interpreted and ultimately displayed in relation to the patient's anatomy for the surgical team to use in conjunction with other tracked anatomical structures and surgical instruments.

Surgical tools developed to facilitate the insertion or extraction of implanted markers have aimed to minimize invasiveness of the use of such tools as well as the associated surgical procedures. However, a problem associated with the use of such implantable marker insertion and extraction tools concerns avoiding causing damage to the cable portion of a marker's sensor assembly. An associated problem concerns the surgical team member's ability to accurately and efficiently operate the marker insertion and extraction tool in the performance of a procedure.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIGS. 1a-1b illustrate generally a clamping mechanism that secures a cable of a sensor in accordance with some embodiments.

FIGS. 2a-2b illustrate generally an alternative clamping mechanism in accordance with some embodiments.

FIG. 3 illustrates generally a partial cross-section perspective view of a tool constructed in accordance with some embodiments.

FIGS. 4a-4b illustrate generally a tool holding a sensor in a first unclamped position and a second clamped position in accordance with some embodiments.

FIG. 5 illustrates generally an insertion tool for locking a cable of a sensor in accordance with some embodiments.

FIG. 6 illustrates an alternative insertion tool for locking a cable of a sensor in accordance with some embodiments.

### DETAILED DESCRIPTION

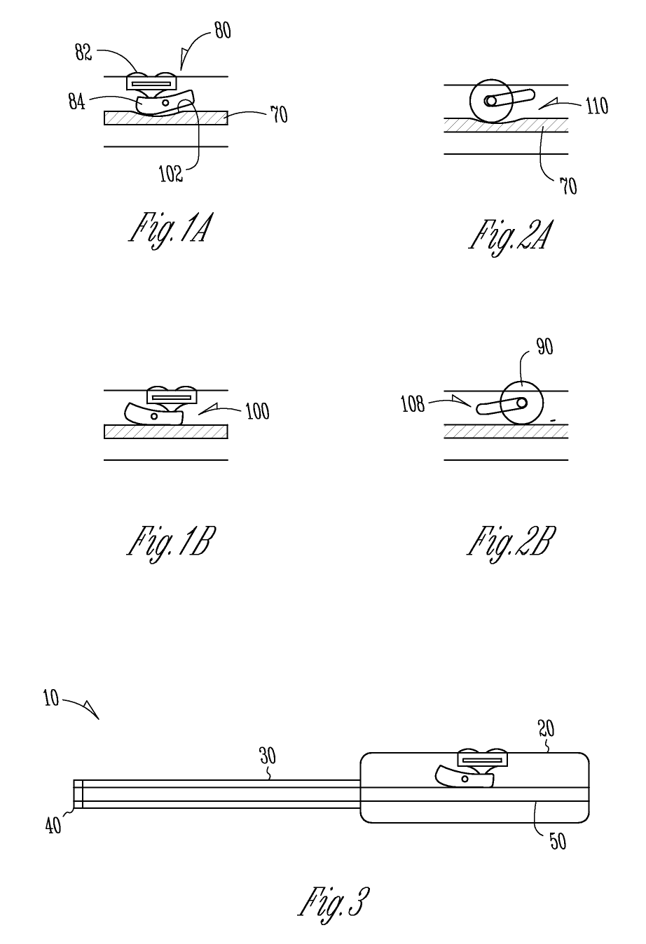

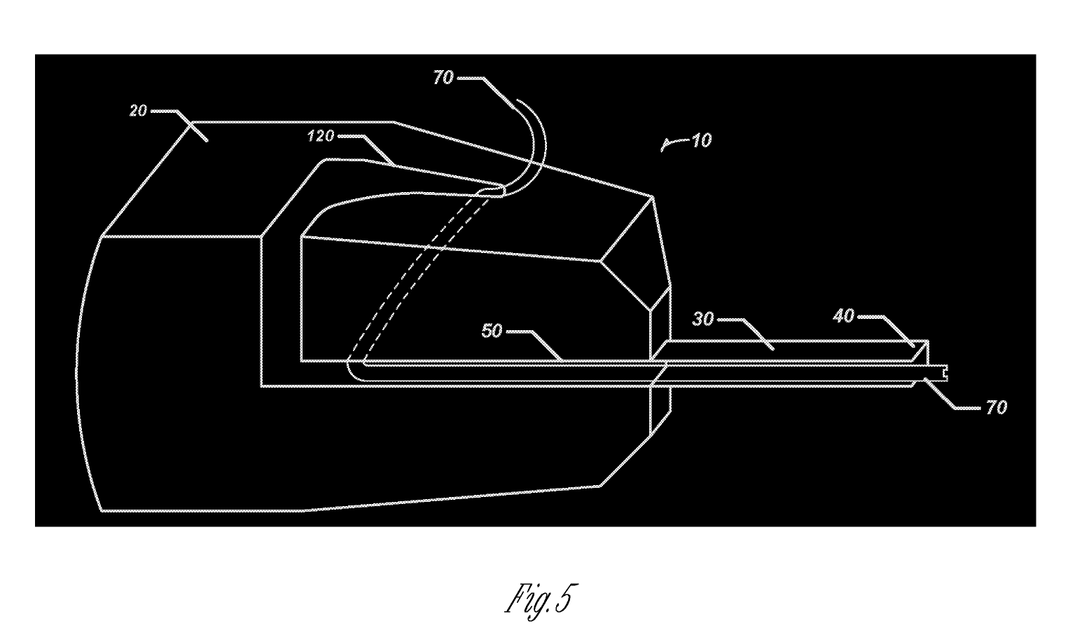

When a member of a surgical team uses a tracking or navigation system with implantable markers, these markers need to be reliably placed and removed with minimal damage to the actual sensor assembly and can be inserted or extracted with a single hand without risk of losing the sensor assembly inside the patient. Referring, now to FIGS. 1-6, the marker insertion or extraction tool 10 comprises a grip portion 20 and an extension portion 30 extending distally from the grip portion 20 to a distal end 40. A slot 50, which can be either curved or straight, that extends the full length of the tool 10.

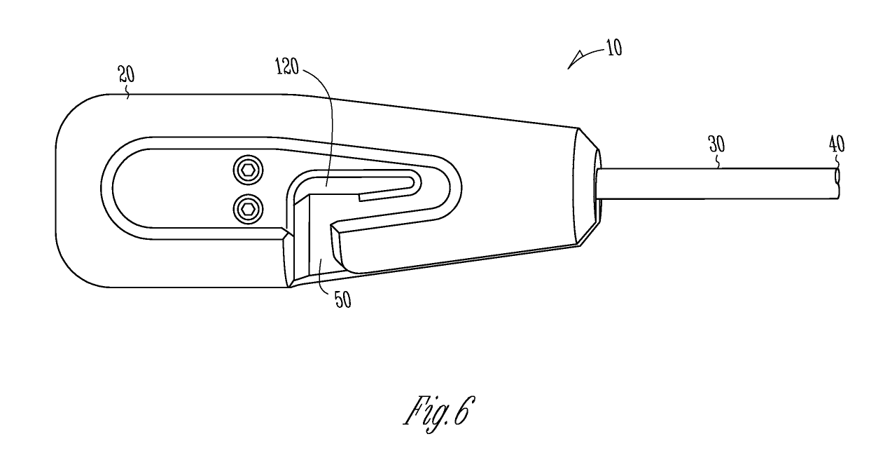

Referring specifically to FIGS. 4a and 4b, the tool 10 optionally has a cavity in the distal end 40 that enables it to be fittingly engaged with a sensor assembly 60, where the sensor assembly 60 includes a head 62. In an embodiment, the cavity has a shape that complementarily matches the shape of the sensor assembly head 62. In a further embodiment, the head 62 is shaped like a bolt and the cavity is matingly shaped like a socket that is fitted to the bolt. In a further embodiment, the sensor assembly 60 is further comprised of an anchoring portion 65 having screw threads, for example, for secure insertion into bone or other biological material and a cable 70 that extends away from the head 62 for communication of position information to a remote computational device. The slot 50 in the tool 10 is sized to snugly accommodate the cable 70 so it does not fall out or get in the way during insertion and removal of the sensor assembly 60. This can be accomplished, for example, through the use of a “V” groove so that the cable is wedged in the slot or other means known in the art. In another embodiment, the slot 50 can include a tapered channel to secure the cable 70. In yet other embodiments, the slot 50 can include inwardly curved lips along the channel opening to retain the cable once inserted (or pressed) into the slot. In still other embodiments, the cross-sectional shape of slot 50 forms a U shape with rounded or squared off corners (FIG. 5 illustrated a slot 50 with a U shaped cross-section with squared off corners).

The grip portion 20 of the tool further provides a clamping mechanism 80 for securing the wire in place within the slot 50, which, in turn, secures the sensor assembly head 62 to the distal end 40 of the sensor assembly via the tension in the wire securing the sensor head in the cavity.

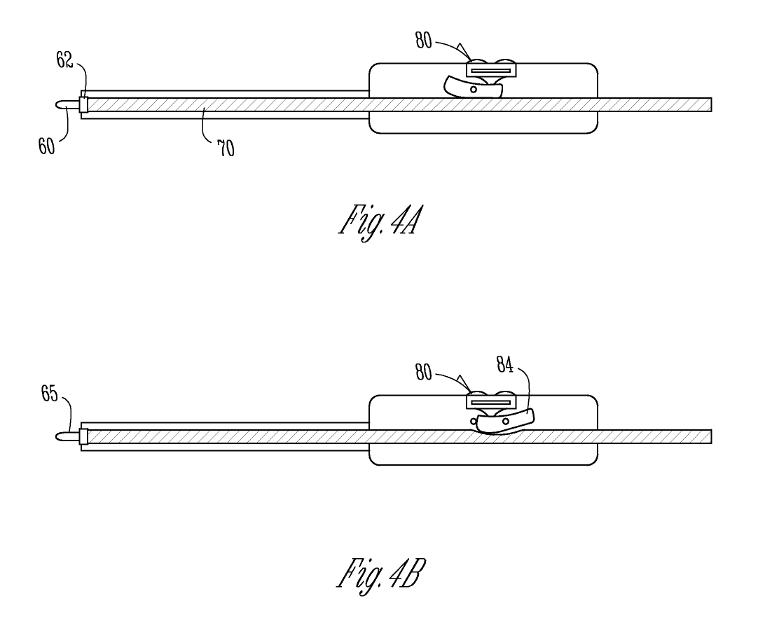

Referring, again, to FIGS. 1a-b, in an embodiment, the clamping mechanism 80 comprises a slide lock 82 configured to optionally depress a locking cam 84 against the cable 70 when slid by the surgeon from an unlocked position 100 to a locked position 102. In an alternative embodiment illustrated by FIGS. 2a-2b, a wheel 90 can be moved along a track from an unlocked position 108 shown in FIG. 2b to a locked position 110 illustrated by FIG. 2a, wherein the locked position clamps the cable 70 in place.

A member of a surgical team can operate the tool to insert or extract a sensor assembly with a single hand. The clamping mechanism can be switched between the locked and the unlocked position with the surgeon's finger, for example, while the tool can be twisted clockwise or counter-clockwise axially in much the same manner as a screwdriver is operated. In an embodiment using press-fit markers, the member of the surgical team need only apply an appropriate amount of pressure on the grip to insert the marker.

FIG. 5 illustrates generally an insertion or extraction tool 10 for locking a cable 70 of a sensor (not shown) in accordance with some embodiments. The insertion or extraction tool 10 comprises a grip portion 20 and an extension portion 30 extending distally from the grip portion 20 to a distal end 40. A slot 50, which can include straight portions, curved portions, turns, or the like, and extends the full length of the tool 10, such as through the extension portion 30 and the grip portion 20. In the embodiment shown in FIG. 5, the slot 50 can include a turn (e.g., 90 degrees, 45 degrees, 135 degrees, or any other degree turn) and exit the grip portion 20 on a side of the grip portion 20. In an embodiment, the turn in the slot 50 can be used to redirect tension through the cable. The insertion or extraction tool 10 shown in FIG. 5 includes a locking slot 120. The locking slot 120 includes a channel that narrows in the direction of the distal end 40 of the extension portion 30. The narrow portion of the channel in the locking slot 120 is sized to produce an interference fit with respect to a diameter of the cable 70. As shown in FIG. 5, the cable 70 is locked when it is pulled under tension through the slot 50 and secured in the narrow portion of the locking slot 120. The tension in the cable 70 can secure a sensor assembly (not shown) to the distal end 40 of the extension portion 30.

The insertion or extraction tool 10 shown in FIG. 5 can be manipulated with a single hand during an operation. For example, the cable 70 can be placed in the slot 50 and attached to a sensor assembly. Tension can then be applied to the cable 70 to secure the sensor assembly to the distal end 40 of the extension portion 30. The cable 70 can be locked using the locking slot 120. The operations described above can be prepared in advance. The cable 70 can also be plugged into a computer, monitor, relay device, electronic interface, etc., to convey information from the sensor assembly, such as position information. At this point, the grip portion 20 can be manipulated to insert the sensor assembly in a bone of a patient. The cable 70 can be released from the locking slot 120 using a single hand, such as by moving a thumb or an articulated digit. Once the cable 70 is unlocked from the locking slot 120, it can be removed from the slot 50. After the cable is removed from the locking slot 120 and the slot 50, the insertion or extraction tool 10 can be removed from the patient, including removing the extension portion 30, leaving the sensor assembly in the patient, and leaving the cable 70 partially in the patient.

The insertion tool 10 illustrated in FIG. 5 is configured to allow the sensor cable 70 to be connected (or plugged in) to a device to receive the sensor signals prior to the operation. The configuration of the slot 50 and locking slot 120 are such that it is not necessary to thread the unconnected end of the cable 70 through any portion of the insertion tool 10. Insertion tools with other locking mechanisms can also be configured to allow for similar operation, provided the entire length of slot 50 is accessible when the mechanism is in an unlocked state. Connecting sensor cables prior to beginning a surgical procedure can shorten the overall procedure time, which is beneficial to both patient and surgeon. Additionally, pre-connecting sensor cables can allow for the markers to be checked for proper function prior to insertion into the patient.

In an embodiment, a portion of the sensor assembly, such as an anchoring portion, can be inserted into a bone of a patient. The anchoring portion can include a press-fit anchor, including a plurality of fins for securing the anchoring portion to the bone. The fins can be used to prevent rotation or lateral movement of the sensor. The anchoring portion can include a biocompatible material, and can include machined, cast, or injection molded portions, such as plastic. In another embodiment, the cable can include one or more wires that can carry different information, power, grounding, etc.

FIG. 6 illustrates an alternative insertion tool for locking a cable of a sensor in accordance with some embodiments. The insertion tool of FIG. 6 can be used similarly to the tool 10 of FIG. 5.

While in accordance with the patent statutes the various alternative embodiments of the instant invention have been described in detail above, it should be understood that various other modifications and alternatives can be envisioned by those persons skilled in the art without departing from either the spirit of the invention or the scope of the appended claims.

### VARIOUS NOTES & EXAMPLES

Each of these non-limiting examples can stand on its own, or can be combined in various permutations or combinations with one or more of the other examples.

Example 1 includes the subject matter embodied by a sensor insertion system comprising: an insertion tool comprising: a grip portion including a clamping mechanism, an extension portion, the extension portion extending distally from the grip portion to a distal end of the insertion tool, and a sensor assembly detachably coupled to the distal end of the extension portion, the sensor assembly including a head portion and an anchoring portion, the anchoring portion may include screw threads and be configured to screw into a bone when torque is applied to the grip portion, a cable running through the extension portion and attached to the sensor assembly, wherein the clamping mechanism is configured to secure the sensor assembly to the distal end of the extension portion, and wherein the grip portion and the extension portion are configured to be detached from the sensor assembly and the cable after the sensor assembly is screwed into the bone.

In Example 2, the subject matter of Example 1 can optionally include wherein the distal end of the extension portion includes a cavity configured to fittingly engage with the head portion of the sensor assembly.

In Example 3, the subject matter of one or any combination of Examples 1-2 can optionally include wherein the head portion includes a bolt shape and the cavity includes a socket shape configured to fit the bolt shape.

In Example 4, the subject matter of one or any combination of Examples 1-3 can optionally include wherein the sensor assembly is configured to convey position information via the cable to a remote device.

In Example 5, the subject matter of one or any combination of Examples 1-4 can optionally include wherein the insertion tool includes a channel running the length of the insertion tool.

In Example 6, the subject matter of one or any combination of Examples 1-5 can optionally include wherein the channel includes a V-groove channel configured to snuggly fit the cable, the V-groove channel running through the grip portion and the extension portion.

In Example 7, the subject matter of one or any combination of Examples 1-6 can optionally include wherein the clamping mechanism includes a slide lock.

In Example 8, the subject matter of one or any combination of Examples 1-7 can optionally include wherein the slide lock is configured to depress a locking cam against the cable when the slide lock is slid from an unlocked position to a locked position.

In Example 9, the subject matter of one or any combination of Examples 1-8 can optionally include wherein the clamping mechanism includes a wheel lock, the wheel lock configured to lock the cable when the wheel lock is moved along a track from an unlocked position to a locked position.

In Example 10, the subject matter of one or any combination of Examples 1-9 can optionally include wherein the clamping mechanism is configured to secure the sensor assembly to the distal end of the extension portion using tension in the cable.

In Example 11, the subject matter of one or any combination of Examples 1-10 can optionally include wherein the insertion tool is configured to be manipulated with a single hand.

Example 12 includes the subject matter embodied by a method for insertion of a sensor assembly comprising: locking a head portion of the sensor assembly to a distal end of an extension portion of an insertion tool using a clamping mechanism in a grip portion of the insertion tool, inserting the sensor assembly into a patient using the insertion tool, twisting the grip portion of the insertion tool to screw an anchoring portion of the sensor assembly into a bone of the patient, wherein the anchoring portion may include screw threads, unlocking the head portion of the sensor assembly from the distal end of the extension portion of the insertion tool using the clamping mechanism, and removing the insertion tool, including the grip portion and the extension portion, leaving the sensor assembly and a cable attached to the sensor assembly in the patient.

In Example 13, the subject matter of Example 12 can optionally include wherein the clamping mechanism includes a slide lock.

In Example 14, the subject matter of one or any combination of Examples 12-13 can optionally include wherein locking the head portion includes sliding the slide lock from an unlocked position to a locked position to depress a locking cam against the cable.

In Example 15, the subject matter of one or any combination of Examples 12-14 can optionally include wherein the clamping mechanism includes a wheel lock.

In Example 16, the subject matter of one or any combination of Examples 12-15 can optionally include wherein locking the head portion includes moving the wheel lock along a track from an unlocked position to a locked position to lock the cable.

In Example 17, the subject matter of one or any combination of Examples 12-16 can optionally include wherein locking the head portion includes securing the sensor assembly to the distal end of the extension portion using tension in the cable.

In Example 18, the subject matter of one or any combination of Examples 12-17 can optionally include wherein the method is performed by a single human hand.

In Example 19, the subject matter of one or any combination of Examples 12-18 can optionally include wherein the method is performed by an articulated arm.

Example 20 includes the subject matter embodied by a sensor insertion system comprising: an insertion tool comprising: a grip portion including a locking slot, an extension portion, the extension portion extending distally from the grip portion to a distal end of the insertion tool, and a sensor assembly detachably coupled to the distal end of the extension portion, the sensor assembly including a head portion and an anchoring portion, the anchoring portion configured to attach to a bone, a cable running through the extension portion and attached to the sensor assembly, wherein the cable is secured to the grip portion of the insertion tool at the locking slot, and wherein the grip portion and the extension portion are configured to be detached from the sensor assembly and the cable after the sensor assembly is attached to the bone.

In Example 21, the subject matter of Example 20 can optionally include wherein the distal end of the insertion tool includes a cavity configured to fittingly engage with the head portion of the sensor assembly.

In Example 22, the subject matter of one or any combination of Examples 20-21 can optionally include wherein the head portion includes a bolt shape and the cavity includes a socket shape configured to fit the bolt shape.

In Example 23, the subject matter of one or any combination of Examples 20-22 can optionally include wherein the sensor assembly is configured to convey position information via the cable to a remote device.

In Example 24, the subject matter of one or any combination of Examples 20-23 can optionally include wherein the cable is attached to the sensor assembly at a sensor end and attached to an electronic interface at an end opposite the sensor end.

In Example 25, the subject matter of one or any combination of Examples 20-24 can optionally include wherein the insertion tool includes a channel running the length of the insertion tool.

In Example 26, the subject matter of one or any combination of Examples 20-25 can optionally include wherein the channel includes a V-groove channel configured to snuggly fit the cable, the V-groove channel running through the grip portion and the extension portion.

In Example 27, the subject matter of one or any combination of Examples 20-26 can optionally include wherein the locking slot includes a channel with a narrow portion to secure the cable.

In Example 28, the subject matter of one or any combination of Examples 20-27 can optionally include wherein the cable is secured to the grip portion of the insertion tool at the locking slot when tension is applied to the cable and the cable is pulled through the locking slot to the narrow portion of the channel.

In Example 29, the subject matter of one or any combination of Examples 20-28 can optionally include wherein the sensor assembly is secured to the distal end of the extension portion using the tension applied to the cable.

In Example 30, the subject matter of one or any combination of Examples 20-29 can optionally include wherein the anchoring portion includes a plurality of fins to attach the anchoring portion in the bone.

In Example 31, the subject matter of one or any combination of Examples 20-30 can optionally include wherein the insertion tool is configured to be manipulated with a single hand.

Example 32 includes the subject matter embodied by a method for insertion of a sensor assembly comprising: locking a cable attached to the sensor assembly to a grip portion of an insertion tool using a locking slot in the grip portion of the insertion tool, wherein locking the cable secures the sensor assembly to a distal end of an extension portion of the insertion tool, securing an anchoring portion of the sensor assembly to a bone of a patient using the insertion tool, unlocking the cable from the locking slot, and removing the insertion tool, including the grip portion and the extension portion, leaving the sensor assembly and at least a portion of the cable in the patient.

In Example 33, the subject matter of Example 32 can optionally include wherein locking the cable includes inserting the cable into a channel with a narrow portion in the locking slot.

In Example 34, the subject matter of one or any combination of Examples 32-33 can optionally include wherein locking the cable includes applying tension to the cable and pulling the cable through a 90 degree turn in the locking slot to the narrow portion of the channel.

In Example 35, the subject matter of one or any combination of Examples 32-34 can optionally include wherein locking the cable includes securing the sensor assembly to the distal end of the extension portion using the applied tension in the cable.

In Example 36, the subject matter of one or any combination of Examples 32-35 can optionally include wherein the anchoring portion includes a plurality of fins to secure the anchoring portion to the bone.

In Example 37, the subject matter of one or any combination of Examples 32-36 can optionally include wherein the method is performed by a single human hand.

In Example 38, the subject matter of one or any combination of Examples 32-37 can optionally include wherein the method is performed by an articulated arm.

Example 39 includes the subject matter embodied by a sensor assembly insertion tool comprising: a grip including a longitudinal slot and a cable locking mechanism, the longitudinal slot extending through at least a portion of the grip, and a distal insertion extension including a continuation of the longitudinal slot extending distally along the entire length of the distal insertion extension from a distal end of the grip to a distal end of the sensor assembly insertion tool, wherein the longitudinal slot is configured to secure a cable attached to a sensor assembly, wherein the cable locking mechanism is configured to releasably lock the cable in a stable position, and wherein the distal end of the sensor assembly insertion tool is configured to hold the sensor assembly in a position to facilitate insertion into a bone.

In Example 40, the subject matter of Example 39 can optionally include wherein the locking mechanism comprises a slide lock.

In Example 41, the subject matter of Example 39 can optionally include wherein the locking mechanism comprises a wheel lock.

In Example 42, the subject matter of Example 39 can optionally include wherein the locking mechanism comprises a taper slot.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations.

What is claimed is:

1. A sensor insertion system comprising:
an insertion tool comprising:
a grip portion including a locking slot;
an extension portion, the extension portion extending distally from the grip portion to a distal end of the insertion tool; and
a sensor assembly detachably coupled to the distal end of the extension portion, the sensor assembly including a head portion and an anchoring portion, the anchoring portion configured to attach to a bone;
a cable running through the extension portion and attached to the sensor assembly, wherein the cable is secured to the grip portion of the insertion tool at the locking slot; and
wherein the grip portion and the extension portion are configured to be detached from the sensor assembly and the cable after the sensor assembly is attached to the bone.

2. The sensor insertion system of claim 1, wherein the distal end of the insertion tool includes a cavity configured to fittingly engage with the head portion of the sensor assembly.

3. The sensor insertion system of claim 2, wherein the head portion includes a bolt shape and the cavity includes a socket shape configured to fit the bolt shape.

4. The sensor insertion system of claim 1, wherein the sensor assembly is configured to convey position information via the cable to a remote device.

5. The sensor insertion system of claim 4, wherein the cable is attached to the sensor assembly at a sensor end and attached to an electronic interface at an end opposite the sensor end.

6. The sensor insertion system of claim 1, wherein the insertion tool includes a channel running the length of the insertion tool.

7. The sensor insertion system of claim 6, wherein the channel includes a V-groove channel configured to snuggly fit the cable, the V-groove channel running through the grip portion and the extension portion.

8. The sensor insertion system of claim 1, wherein the locking slot includes a channel with a narrow portion to secure the cable.

9. The sensor insertion system of claim 8, wherein the cable is secured to the grip portion of the insertion tool at the locking slot when tension is applied to the cable and the cable is pulled through the locking slot to the narrow portion of the channel.

10. The sensor insertion system of claim 9, wherein the sensor assembly is secured to the distal end of the extension portion using the tension applied to the cable.

11. The sensor insertion system of claim 1, wherein the anchoring portion includes a plurality of fins to attach the anchoring portion in the bone.

12. The sensor insertion system of claim 1, wherein the insertion tool is configured to be manipulated with a single hand.

13. A method for insertion of a sensor assembly comprising:
locking a cable attached to the sensor assembly to a grip portion of an insertion tool using a locking slot in the grip portion of the insertion tool, wherein locking the cable secures the sensor assembly to a distal end of an extension portion of the insertion tool;
securing an anchoring portion of the sensor assembly to a bone of a patient using the insertion tool;
unlocking the cable from the locking slot; and
removing the insertion tool, including the grip portion and the extension portion, leaving the sensor assembly and at least a portion of the cable in the patient.

14. The method of claim 13, wherein locking the cable includes inserting the cable into a channel with a narrow portion in the locking slot.

15. The method of claim 14, wherein locking the cable includes applying tension to the cable and pulling the cable through a 90 degree turn in the locking slot to the narrow portion of the channel.

16. The method of claim 15, wherein locking the cable includes securing the sensor assembly to the distal end of the extension portion using the applied tension in the cable.

17. The method of claim 13, wherein the anchoring portion includes a plurality of fins to secure the anchoring portion to the bone.

18. A sensor assembly insertion tool comprising:
a grip including a longitudinal slot and a cable locking mechanism, the longitudinal slot extending through at least a portion of the grip; and
a distal insertion extension including a continuation of the longitudinal slot extending distally along the entire length of the distal insertion extension from a distal end of the grip to a distal end of the sensor assembly insertion tool;
wherein the longitudinal slot is configured to secure a cable attached to a sensor assembly, wherein the cable locking mechanism is configured to releasably lock the cable in a stable position, and wherein the distal end of the sensor assembly insertion tool is configured to hold the sensor assembly in a position to facilitate insertion into a bone.

19. The sensor assembly insertion tool of claim 18, wherein the locking mechanism comprises a slide lock.

20. The sensor assembly insertion tool of claim 18, wherein the locking mechanism comprises a wheel lock.

21. The sensor assembly insertion tool of claim 18, wherein the locking mechanism comprises a taper slot.
